(19) United States
(12) Patent Application Publication
(10) Pub. No.: US 2010/0178307 A1
(43) Pub. Date: Jul. 15, 2010

(54) TRANSDERMAL ANTI-DEMENTIA ACTIVE AGENT FORMULATIONS AND METHODS FOR USING THE SAME

(76) Inventors: Jianye Wen, Palo Alto, CA (US); Yoshiko Katori Stowell, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/687,034

(22) Filed: Jan. 13, 2010

(51) Int. Cl. A61K 9/70; A61K 31/445; A61P 25/18

(52) U.S. Cl. 424/400; 514/319

(57) ABSTRACT

Transdermal anti-dementia active agent systems are provided. Aspects of systems include a transdermal preparation, first overlay and second overlay. Also provided are methods of using the systems, e.g. for administering an anti-dementia active agent to a subject.

Alzheimer's disease is a degenerative brain disease that causes dementia, a progressive decline in cognitive function beyond what might be expected from normal aging. Short-term memory loss is the most common symptom, and later symptoms include confusion, anger, mood swings, language breakdown, long-term memory loss, and the general withdrawal of the subject as his or her senses decline. Alzheimer's disease has no current cure, however its symptoms can be treated with active agents, such as acetylcholinesterase inhibitors (e.g., donepezil, galantamine, rivastigimine, tacrine, etc.) and N-methyl D-aspartate (NMDA) receptor antagonists (e.g., memantine).

Donepezil, known chemically as (±)-2,3-dihydro-5,6-dimethoxy-2,1-(phenylmethyl)-4-piperidinyl]methyl]-1H-inden-1-one, is a reversible acetylcholinesterase inhibitor that is used to treat the symptoms of Alzheimer's disease. Typically, donepezil is provided as donepezil hydrochloride in tablet form for oral administration (e.g., Aricept®, Eisai Co., Ltd., Japan).

Transdermal active agent formulations, also known as transdermal patches or skin patches, are adhesive patches containing an active agent that are placed on the skin to deliver the active agent through the skin. Transdermal patches deliver the active agent by percutaneous absorption, which is the absorption of substances through unbroken skin. After a transdermal patch is applied to the skin, the active agent contained in the patch passes through, or permeates the skin and can reach its site of action through a systemic blood flow. Alternatively, the transdermal patch may be placed on the desired treatment site such that the medication contained in the patch is delivered topically.

### SUMMARY

Transdermal anti-dementia active agent systems are provided. Aspects of systems include a transdermal preparation, first overlay and second overlay. Also provided are methods of using the systems, e.g. for administering an anti-dementia active agent to a subject.

### BRIEF DESCRIPTION OF THE FIGURES

FIG. 1 shows a cross sectional view of an embodiment of the transdermal active agent formulation described herein.

FIG. 2 shows an overhead view of the first overlay according to an embodiment of the invention.

FIG. 3 shows an overhead view of the first overlay according to an embodiment of the invention.

FIG. 4 shows an assembled system of the invention, in which a transdermal preparation is covered by a first overlay, which in turn is covered by a second overlay.

### DETAILED DESCRIPTION

Transdermal anti-dementia active agent systems are provided. Aspects of systems include a transdermal preparation, first overlay and second overlay. Also provided are methods of using the systems, e.g. for administering an anti-dementia active agent to a subject.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be constructed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various embodiments of the invention, aspects of the transdermal active agent systems are reviewed first in greater detail, followed by a detailed description of methods of using the systems and a review of kits that include the systems.

### Transdermal Anti-Dementia Active Agent Systems

As summarized above, transdermal anti-dementia active agent systems are provided. Systems of the invention are configured to deliver an anti-dementia active agent to a subject when topically applied to a skin surface of a subject. Transdermal active agent systems including the following components: (a) a transdermal preparation; (b) a first overlay; and (c) a second overlay. Each of these components is now reviewed separately in greater detail.

### Transdermal Preparation

Transdermal preparations of the invention may have one or more layers, where a formulation having multiple layers is referred to herein as a formulation that includes a multilaminate design. Transdermal preparations may have at least an active agent reservoir layer that includes an anti-dementia active agent. In some instances, the transdermal preparations are single layer compositions that only include the active agent reservoir layer. In yet other instances, the transdermal preparation may include additional layers, such as one or more of a backing layer, an adhesive layer, an intermediate layer, a release liner, etc.

### Active Agent Reservoir Layer

The active agent reservoir layer contains an amount of an anti-dementia active agent. In some instances, the anti-dementia active agent is present in the reservoir layer in an amount ranging from 0.5-50% by weight, such as 10-40% by weight, and including 15-35% by weight.

Anti-dementia active agents of interest include, but are not limited to donepezil, galantamine, rivastigimine, tacrine, memantine, and analogues thereof (where a given active agent (e.g., donepezil or analogue thereof) may be present as a free base or salt).

In some embodiments, the anti-dementia active agent is donepezil. Donepezil free base has the empirical formula of C₂₄H₂₉NO₃ and the IUPAC name (±)-2,3-dihydro-5,6-dimethoxy-2-[[1-(phenylmethyl)-4-piperidinyl]methyl]-1H-inden-1-one. Donepezil has the following chemical structure:

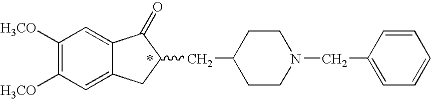

Salts of donepezil may include the hydrochloride salt, and the like. Donepezil hydrochloride salt, or donepezil-HCl, has the empirical formula of C₂₄H₂₉NO₃.HCl and the IUPAC name (±)-2,3-dihydro-5,6-dimethoxy-2-[[1-(phenylmethyl)-4-piperidinyl]methyl]-1H-inden-1-one hydrochloride. Donepezil-HCl has the following chemical structure:

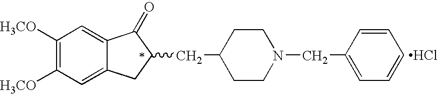

As indicated above, the anti-dementia active agent is present in the active agent reservoir layer as a free base and/or a salt, such as but not limited to donepezil free base and/or donepezil-HCl. In some instances, the active agent present in the active agent reservoir layer is donepezil-HCl.

In addition to the anti-dementia active agent, the active agent reservoir layer may include a number of different additional components. Additional components that may be present in the active agent reservoir layers include, but are not limited to: stability enhancers and/or flux modulators (e.g., aminated polymers), plasticizers (e.g., carboxylic acid esters), percutaneous absorption enhancers, (e.g., carboxylic acid esters); an active agent stabilizer (e.g., polyhydric alcohol); and a physicochemical stabilizer (e.g., acrylic polymer). In some instances, the active agent reservoir layer includes an aminated polymer; a carboxylic acid ester; a polyhydric alcohol; and an acrylic polymer.

The aminated polymer in the active agent reservoir layer may be a copolymer which may include a dialkylaminoalkyl(meth)acrylate and a monomer unit selected from an alkyl(meth)acrylate, a hydroxyalkyl(meth)acrylate and a combination thereof. The dialkylaminoalkyl(meth)acrylate may be a di-C₁₋₄ alkylamino C₁₋₁₂ alkyl(meth)acrylate, such as a di-C₁₋₂ alkylamino C₁₋₂ alkyl(meth)acrylate. Dialkylaminoalkyl(meth)acrylates of interest include, but are not limited to: dimethylaminomethyl(meth)acrylate, diethylaminomethyl (meth)acrylate, dimethylaminoethyl(meth)acrylate, dimethylaminobutyl(meth)acrylate, diethylaminooctyl (meth)acrylate, and the like. The monomer units other than the dialkylaminoalkyl(meth)acrylate in the copolymer may be alkyl (meth)acrylates or a hydroxyalkyl(meth)acrylates, such as C₁₋₁₂ alkyl(meth)acrylates or monohydroxy C₂₋₄ alkyl (meth)acrylates, and including C₁₋₄ alkyl(meth)acrylates or a monohydroxy C₂₋₄ alkyl(meth)acrylates. Monomer units of interest include, but are not limited to: methyl(meth)acrylate, ethyl (meth)acrylate, propyl(meth)acrylate, butyl(meth)acrylate, octyl (meth)acrylate, 2-hydroxyethyl(meth)acrylate, 2-ethylhexyl (meth)acrylate, dodecyl(meth)acrylate, and the like. In some instances, the aminated polymer is a copolymer which includes a di-C₁₋₂ alkylamino C₁₋₂ alkyl (meth)acrylate and a monomer unit selected from a C₁₋₄ alkyl (meth)acrylate, a monohydroxy C₂₋₄ alkyl(meth)acrylate and a combination thereof, such as a methyl(meth)acrylate-butyl (meth)acrylate-dimethylaminoethyl(meth)acrylate copolymer, and including a methyl methacrylate-butyl methacrylate-dimethylaminoethyl methacrylate copolymer. Methyl methacrylate-butyl methacrylate-dimethylaminoethyl methacrylate copolymer is commercially available, for example, as the EUDRAGIT® E100 (Degussa) copolymer. In some instances, the amount of the aminated polymer in the active agent reservoir layer may range from 5-30% by weight, such as 10-30% by weight.

The active agent reservoir may include one or more carboxylic acid esters, which may serve as plasticizers and/or percutaneous absorption enhancers. Carboxylic acid esters of interest include esters of a polyvalent carboxylic acid and a monohydroxy alcohol and esters of a fatty acid and a polyhydric alcohol. In some instances, the active agent reservoir layer includes a combination of these types of esters, such that the reservoir layer includes both an ester of a polyvalent carboxylic acid and a monohydroxy alcohol and an ester of a fatty acid and a polyhydric alcohol. While the total amount of carboxylic acid ester in the active agent reservoir layer may vary, in some instances the total amount of the one or more carboxylic acid esters in the active agent reservoir layer ranges from 3-20%, such as from 5-15% by weight.

Esters of interest include esters of polyvalent carboxylic acids and monohydroxy alcohols, which esters may find use as plasticizing agents in the active agent reservoir layers. The polyvalent carboxylic acid in the ester described may vary, and in some instances is di- or tri-valent. Polyvalent carboxylic acids of interest include those that are C₆₋₁₀. Similarly, the monohydroxy alcohol in the ester may vary, an in some instances is a C₂₋₄ monohydroxy alcohol. Specific esters of polyvalent carboxylic acids and the monohydroxy alcohols of interest include alkyl citrate esters and/or an alkyl sebacate esters, such as C₂₋₄ alkyl citrates and/or C₂₋₄ alkyl sebacates, including tri-(C₂₋₄)-alkyl citrates and/or di-(C₂₋₄-alkyl sebacates, e.g., triethyl citrate and/or diethyl sebacate. While the amount of esters of polyvalent carboxylic acids and the monohydroxy alcohols may vary, in some instances the amount of these types of esters in the active agent reservoir layer ranges from 3-15% by weight, such as from 3-10% by weight.

Esters of interest also include percutaneous absorption enhancers. Examples of esters having functionality as percutaneous absorption enhancers include esters of fatty acid and a polyhydric alcohol, including but not limited to: sorbitan fatty acid esters, propylene glycol fatty acid esters and glycerin fatty acid esters. Of interest in some instances are sorbitan fatty acid esters, such as sorbitan C₇₋₁₉ fatty acid esters. Specific examples of sorbitan fatty acid esters of interest include, but are not limited to: sorbitan monolaurate, sorbitan monostearate, sorbitan monoleate, sorbitan monopalmitate, sorbitan trioleate, and sorbitan tristearate, preferably sorbitan monolaurate. While the amount of the ester of a fatty acid and a polyhydric alcohol may vary, in some instances the amounts of these types of esters in the active agent reservoir layer ranges from 1-10% by weight, such as from 2-5% by weight.

Instead of or in addition to a carboxylic acid ester percutaneous absorption agent, other types of percutaneous absorption agents may be present. Examples of other types of percutaneous absorption agents of interest that may be present include, but are not limited to, those described in U.S. patent application Ser. Nos. 12/437,403 and 12/551,231; the disclosures of which are herein incorporated by reference.

As summarized above, the active agent reservoir layer may also include a stabilizing agent, such as a polyhydric alcohol. Polyhydric alcohols of interest include, but are not limited to: sugar alcohols and/or glycols, such as tritols, pentitols, hexitols, and glycols. Polyhydric alcohol include glycerin, propylene glycol, dipropylene glycol, butylene glycol, d-sorbitol, xylitol, mannitol, polyethylene glycol, and a combination thereof. While the amount of the polyhydric alcohol in the active agent reservoir layer may vary, in some instances the amount ranges 1-20% by weight, such as from 3-15% by weight, including 5-10% by weight.

Also present in the active agent reservoir layer may be an acrylic polymer. Acrylic polymers of interest include (meth)acrylate-vinyl ester copolymers. The (meth)acrylate which is a component of the acrylic polymer may include an alkyl(meth)acrylate, a monohydroxyalkyl(meth)acrylate or an epoxyalkyl(meth)acrylate, such as a C₁₋₁₂ alkyl(meth)acrylate, a monohydroxy C₂₋₄ alkyl(meth)acrylate, or glycidyl(meth)acrylate in some instances, the (meth)acrylate includes methyl(meth)acrylate, ethyl (meth)acrylate, propyl(meth)acrylate, butyl(meth)acrylate, octyl (meth)acrylate, hydroxyethyl(meth)acrylate, 2-ethylhexyl (meth)acrylate, dodecyl(meth)acrylate, glycidyl(meth)acrylate, and the like. The vinyl ester which is a component of the acrylic polymer may vary, and in some instances includes vinyl acetate, vinyl propionate, vinyl butyrate, vinyl crotonate, vinyl caprate and the like, preferably vinyl acetate. In certain embodiments, the acrylic polymer described above is a copolymer composed of a monomer unit selected from an alkyl(meth)acrylate, a monohydroxyalkyl(meth)acrylate, an epoxyalkyl(meth)acrylate, and a combination thereof, and vinyl acetate. Of interest are copolymers that are composed of a monomer unit selected from a C₁₋₁₂ alkyl(meth)acrylate, a monohydroxy C₂₋₄ alkyl(meth)acrylate, glycidyl(meth)acrylate, and a combination thereof, and vinyl acetate. Copolymers of interest may be composed of a monomer unit selected from 2-ethyl hexyl(meth)acrylate, hydroxyethyl(meth)acrylate, glycidyl(meth)acrylate, and a combination thereof, and vinyl acetate. In certain embodiments, the copolymer is composed of a monomer unit selected from 2-ethylhexyl acrylate, hydroxyethyl acrylate, glycidyl methacrylate, and vinyl acetate. Specific examples of the acrylic polymers of interest include, but are not limited to: DURO-TAK®387-2516, 87-2287, 87-4287 copolymers (National Starch & Chemical Co., Ltd.), and the like. When present, the amount of acrylic polymer in the active agent reservoir layer may range from 5-60% by weight, such as 5-50% by weight.

Additional details regarding active agent reservoir layers of interest that include an aminated polymer, one or more carboxylic acid esters, a polyhydric alcohol and an acrylic polymer may be found in United States Published Patent Application 20070259028; the disclosure of which application is herein incorporated by reference. Also of interest are those donepezil transdermal preparations described in U.S. patent application Ser. Nos. 12/437,403 and 12/551,231; the disclosures of which are herein incorporated by reference.

The thickness of the active agent reservoir layer may vary, and in some instances ranges from 50-150 p.m.

The substrate layer may be pressure sensitive adhesive. The terms “pressure-sensitive adhesive”, “self adhesive”, and “self-stick adhesive” mean an adhesive that forms a bond when pressure is applied to adhere the adhesive with a surface. Typically, no solvent, water, or heat is needed to activate the adhesive. For pressure-sensitive adhesives, the degree of bond strength is proportional to the amount of pressure that is used to apply the adhesive to the surface.

Topical preparations may be made up solely of an active agent reservoir layer. However, in some instances, the topical preparations may further include on or more additional layers, such as but not limited to: an adhesive layer, an intermediate layer, a backing layer and a release liner.

### Adhesive Layer

Accordingly, in some instances the topical preparations include an adhesive layer. The transdermal active agent formulation that is employed herein may have an adhesive layer for facilitating adhesion of the transdermal patch to the skin of the subject. When present, the adhesive layer is positioned relative to the active agent reservoir layer such that, upon topical application, it is positioned between the skin surface of a subject and the active agent reservoir layer. In certain embodiments, the adhesive layer may be provided on the active agent reservoir layer. In other cases, an intermediate layer (such as a rate-controlling membrane or a non-rate controlling layer as described in greater detail below) may be provided between the active agent reservoir layer and the adhesive layer.

In some cases, the adhesive layer may be an acrylic pressure-sensitive adhesive layer. In certain embodiments, the acrylic pressure-sensitive adhesive is a copolymer of an acrylate and at least one other monomer, e.g., vinyl acetate, butyl acrylate, 2-ethylhexyl acrylate, hydroxyethyl acrylate, t-octyl acrylamide, methyl methacrylate, and acrylic acid or (meth) acrylic acid. In certain cases, the acrylic pressure-sensitive adhesive may be an acrylate-vinyl acetate copolymer, in an organic solvent solution. Examples of polyacrylate-based adhesives are as follows, identified as product numbers, manufactured by National Starch (DURO-TAK® is a trademark of National Starch adhesives): 87-4098, 87-2516, 87-2051, 87-2052, 87-2054, 87-2196, 87-9259, 87-9261, 87-2979, 87-2510, 87-2353, 87-2100, 87-2852, 87-2074, 87-2258, 87-9085, 87-9301 and 87-5298. DURO-TAK® 87-2287 and 87-4287 both are polymeric adhesives derived from monomer compositions that are similar.

Alternatively, the adhesive composition of this invention may contain polyisobutylene (PIB). PIB is typically a blend of high molecular weight PIB and low molecular weight PIB. As an example, in one effective embodiment the PIB adhesive includes 8 wt % high molecular weight (such as OPPANOL L80, L100, and L140 from BASF) PIB material and 92 wt % low molecular weight (Such as OPPANOL B10, B11, B12, and B13 from BASF) PIB material. The PIB can be with or without tackifiers or plasticizers, such as low molecular weight polybutene (e.g., INDOPOL H1900 and/or high Tg, low molecular weight aliphatic resins such as the ESCOREZ resins available from Exxon Chemical, and the like).

Another kind of adhesive that can be used is a silicone adhesive. The silicone adhesives that may be used are typically high molecular weight polydimethyl siloxanes or polydimethyldiphenyl siloxanes. Formulations of silicone adhesives that are useful in transdermal patches are described in U.S. Pat. Nos. 5,232,702, 4,906,169 and 4,951,622. One example of such a silicone adhesive is Silicone 4202 polydimethylsiloxane adhesive from Dow Corning.

In some instances, the adhesive layer includes one or more components in common with the active agent reservoir layer. Of interest are adhesive layers that include the same acrylic copolymer and/or carboxylic acid ester(s). In some instances, the adhesive layer is made up of the same acrylic copolymer, the ester of a polyvalent carboxylic acid and a monohydroxy alcohol and the ester of a fatty acid and a polyhydric alcohol as found in the active agent reservoir layer.

While the thickness of the adhesive layer may vary, in some instances the thickness ranges from 50 to 100 μm.

### Intermediate Layer

In some cases, the transdermal formulations may have an intermediate layer provided between the active agent reservoir layer and the adhesive layer. In some embodiments, the intermediate layer may be a rate-controlling membrane layer. “Rate-controlling” means that the membrane meters the quantity of active agent that is administered through the skin for a prolonged period of time, such that the active agent is released from the transdermal formulation at a substantially constant rate until the desired total quantity (i.e., target dosage) of active agent is administered.

In certain embodiments, the rate-controlling membrane may be a microporous membrane having pores that allow permeation of the active agent. In these embodiments, the flux or release rate of the active agent by the membrane is controlled by the rate of which the active agent is able to diffuse through the pores of the membrane. The rate-controlling membrane may be any porous material that permits the permeation of the active agent, such as but not limited to polypropylene, polyethylene, polyacrylonitrile, polytetrafluoroethylene, polydimethylsiloxane, polymethyl methacrylate, and combinations thereof. Additionally, the rate-controlling membrane may be single layer or multi-layer (i.e., having one or more microporous membrane layers composed of the same or different material laminated together). In certain embodiments, the rate-controlling membrane is a monolayer polypropylene membrane.

The porosity, pore size and thickness of the rate-controlling membrane depend on the physicochemical properties, such as the molecular weight of the active agent, the flux required, and the like. For example, the rate-controlling membrane may typically have the following properties: a porosity ranging from about 10% to 85%, including from about 20% to 75%, such as from 30% to 50%; a pore size ranging from 0.03-0.25 μm×μm, including 0.03-0.2 μm×μm, such as 0.04-0.12 μm×μm; and a thickness ranging from 10 μm to 70 μm, including from 15 μm to 60 μm, such as from 20 μm to 50 μm. In certain embodiments, the rate-controlling membrane may have a porosity of 37%, a pore size of 0.04-0.12 μm×μm, and a thickness of 25 μm.

In other embodiments, the intermediate layer may be a non-rate controlling layer. “Non-rate controlling” means that the layer does not significantly affect the flux or the release of the active agent from the transdermal formulation. In certain embodiments, the non-rate controlling layer may facilitate the reduction of cold flow (i.e., the movement of material over a period of time) of the layers of the transdermal formulation. In these embodiments, the non-rate controlling layer may be a non-woven layer, such as but not limited to non-woven polyester fabric from Reeway inc., and combinations thereof.

### Backing Layer

The transdermal preparation that is employed herein may have a backing layer. The backing may be flexible to an extent that it can be brought into close contact with a skin surface. The backing is such that it does not absorb the active agent, and does not allow the active agent to be released from the backing side. The backing may include, but is not limited to, non-woven fabrics, fabrics, films (including sheets), porous bodies, foamed bodies, paper, composite materials obtained by laminating a film on a non-woven fabric or fabric, and combinations thereof.

Non-woven fabric may include, but is not limited to the following: polyolefin resins such as polyethylene and polypropylene; polyester resins such as polyethylene terephthalate, polybutylene terephthalate and polyethylene naphthalate; and besides rayon, polyamide, poly(ester ether), polyurethane, polyacrylic resins, polyvinyl alcohol, styrene-isoprene-styrene copolymers, and styrene-ethylene-propylene-styrene copolymers; and combinations thereof. Fabric of interest include, but are not limited to cotton, rayon, polyacrylic resins, polyester resins, polyvinyl alcohol, and combinations thereof.

The film may include, but is not limited to the following: polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof.

The paper may include, but is not limited to impregnated paper, coated paper, wood free paper, Kraft paper, Japanese paper, glassine paper, synthetic paper, and combinations thereof. Composite materials may include, but are not limited to composite materials obtained by laminating the above-described film on the above-described non-woven fabric or fabric.

### Release Liner

In some embodiments, a release liner is provided on the adhesive layer, specifically on a surface of the adhesive layer that is distal from the reservoir layer. The release liner facilitates the protection of the active agent reservoir layer and the adhesive layer. Prior to application onto a skin surface, the release liner may be removed, thereby exposing the adhesive layer. The release liner may be prepared by treating one side of polyethylene-coated wood free paper, polyolefin-coated glassine paper, a polyethylene terephthalate (polyester) film, a polypropylene film, or the like with a silicone treatment.

### Transdermal Preparation Format

As summarized above, topical preparations of interest may have a variety of different formats, which may vary depending on a number of factors, including but not limited to which layers are present, the nature of the active agent to be delivered, etc.

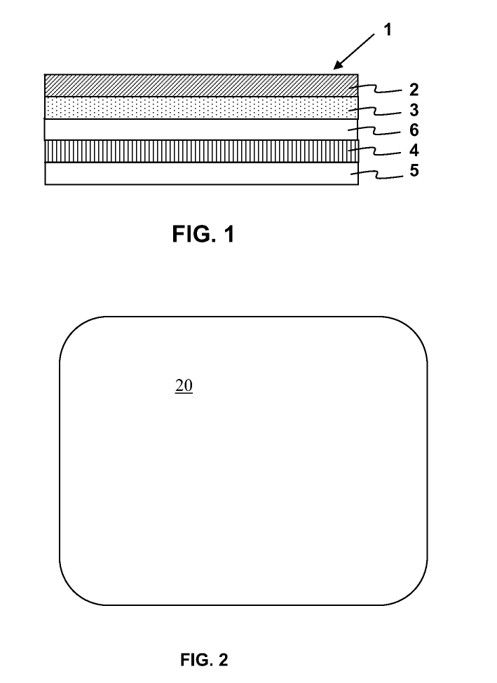

FIG. 1 shows an embodiment of the transdermal active agent preparation 1, where the transdermal active agent preparation 1 includes a backing layer 2, an active agent reservoir layer 3, an adhesive layer 4, and a release liner 5. Also shown is an intermediate layer 6 positioned between the adhesive layer 4 and active agent reservoir layer 3. In these embodiments, the intermediate layer may be a rate-controlling membrane or a non-rate controlling layer.

The size (i.e., area) of the transdermal preparation depends on the transdermal flux rate of the active agent and the target dosage. For example, if the transdermal flux is 4.8 μg/cm²/hr and the target dosage is 5 mg/day, then the transdermal preparation would have an area of about 43 cm². Or for example, if the transdermal flux is 4.8 μg/cm²/hr and the target dosage is 10 mg/day, then the transdermal preparation would have an area of about 87 cm². While the size of a given transdermal preparation may vary, in certain embodiments the preparation is configured to cover a skin site having an area that ranges from 1 to 200 cm², such as from 3 to 100 cm², including 3 to 50 cm², e.g., 3 to 25 cm².

Topical preparations may be configured to provide for a skin permeation rate (i.e., transdermal flux rate) sufficient to administer a target dosage of active agent to a subject over a period of time. In some cases, the target dosage of the active agent may be 5 mg/day or greater over a one week period (i.e., 7 days or 168 hours), including 10 mg/day or greater over one week, such as 15 mg/day or greater over one week. In some cases the maximal skin permeation rate of the active agent may be about 2.5 μg/cm²/hr or greater, including about 4.5 μg/cm²/hr or greater, or about 6.0 μg/cm²/hr or greater, such as about 6.5 μg/cm²/hr or greater. Transdermal flux rates may be determined using the procedure described in examples.

### Fabrication of Transdermal Preparation

Transdermal preparations of systems of the invention may be prepared using any convenient protocol. In some embodiments, an adhesive mass solution obtained by mixing the constituent materials of the active agent reservoir layer is first coated on a liner. Next, the adhesive mass solution is dried at a sufficient temperature, e.g., 70-80° C., to obtain the active agent reservoir layer, on which a backing layer is laminated. Next, an adhesive mass solution which is composed of the materials comprising the adhesive layer, is coated on a liner and dried at a sufficient temperature, e.g., 70-80° C., on which the intermediate membrane is further laminated. The liner of the active agent reservoir layer is then peeled off, and the active agent reservoir layer is then laminated on a surface opposite to the adhesive layer on the drug intermediate membrane to yield a transdermal preparation.

To the adhesive mass solution used for the preparation of the active agent reservoir layer and the adhesive layer may be appropriately added an organic solvent in addition to the constituent materials of the solution. The organic solvent includes, for example, ethyl acetate, butyl acetate, toluene, n-hexane, tetrahydrofuran, dimethylformamide, methanol, ethanol, and the like.

Additional details regarding fabrication protocols of interest may be found in United States Published Patent Application 20070259028; as well as in U.S. patent application Ser. Nos. 12/437,403 and 12/551,231; the disclosures of which are herein incorporated by reference.

### First Overlay

As summarized above, systems of the invention also include a first overlay. In some instances, the overlay is used to increase the adhesion of the composition when applied to the skin. First overlays can include a layer of adhesive present on a backing material, such as a porous, non-porous, foam (e.g., closed cell foam), occlusive, or breathable backing material. In some instances, the first overlay is a closed cell polyolefin foam overlay comprising a pressure sensitive acrylate adhesive. The dimensions of the first overlay are chosen to provide the desired functionality, where the first overlay is generally configured to at least partially cover the transdermal preparation upon topical application. In some instances the dimensions are chosen such that the first overlay, when applied over the transdermal preparation, extends some distance beyond one or more of the sides of the transdermal preparation. In some instances, the area of the adhesive overlay exceeds the area of the transdermal preparation by 5% or more, such as by 10% or more, including by 20% or more. An overhead view of a first overlay according to an embodiment of the invention is provided in FIG. 2.

### Second Overlay

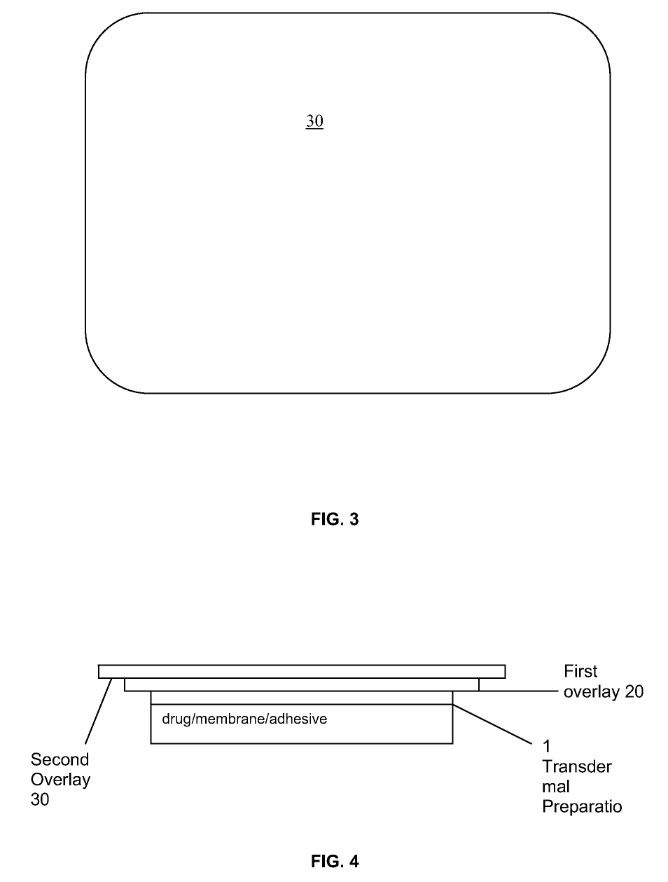

As summarized above, systems of the invention also include a second overlay. In some instances, the second overlay is used to increase the adhesion of transdermal preparation when applied to the skin. Second overlays can include a layer of adhesive present on a backing material, such as a porous, non-porous, occlusive, or breathable backing material. The second overlay structure is a single-coated polyurethane medical tape on a white carrier. In some instances, the second overlay is a single-coated polyurethane medical tape on a carrier. The dimensions of the second overlay are chosen to provide the desired functionality, where the second overlay is generally configured to at least partially cover the first overlay upon topical application. In some instances the dimensions are chosen such that the second overlay, when applied over the first overlay which is applied over the topical preparation, extends some distance beyond one or more of the sides of the first overlay. In some instances, the area of the second overlay exceeds the area of the first overlay by 5% or more, such as by 10% or more, including by 20% or more. An overhead view of a second overlay according to an embodiment of the invention is provided in FIG. 3.

### Methods

Methods for administering an anti-dementia agent to a subject are also provided. Aspects of the methods may include applying to a skin site of a subject a transdermal anti-dementia active agent system as described in detail above, and maintaining the system at the skin site of the subject for a period of time sufficient to deliver the active agent to the subject. The transdermal active agent system may be applied to the skin of the subject, for example at a skin site, a keratinized skin site, etc. The transdermal active agent system may be applied to a skin surface such that the formulation is adhered to a skin surface by the adhesion of the adhesive layer to the skin surface.

Application of the system to the skin site may include a number of different steps, e.g., depending on the particular configuration of the transdermal preparation and system. In some instances, the application protocol includes a step of removing a release liner from the transdermal preparation, e.g., to expose the adhesive layer or active agent reservoir layer (if an adhesive layer is not present). Next, the preparation is contacted to the skin site in a manner such that the backing side of the preparation is furthest away from the skin surface, e.g., so that the adhesive layer (or active agent reservoir layer if no adhesive layer is present) contacts the skin surface. Following topical application of the transdermal preparation, the first overlay is applied over the transdermal preparation in a manner such that the first overlay at least partially covers the transdermal preparation (e.g., at 30% or more, 50% or more, 75% or more etc). In some instances, the first overlay is applied in a manner sufficient to completely cover the transdermal preparation. Next, the second overlay is applied over the first overlay in a manner such that the second overlay at least partially covers the first overlay (e.g., at 30% or more, 50% or more, 75% or more etc). In some instances, the second overlay is applied in a manner sufficient to completely cover the first overlay. An example of an illustrated assembled system of the invention is provided in FIG. 4. Of course the order of the above steps may be altered as desired. For example, a composite structure of the transdermal preparation, first and second overlays may be produced first, followed by removal of the release liner and then topical application of the system.

In some cases, the transdermal active agent formulation may be applied to a skin site for an amount of time sufficient to deliver the active agent to the subject. In some cases, the transdermal active agent formulation may be applied to the skin site for an amount of time sufficient to deliver an effective amount of the active agent to the subject. The term “effective amount” means a dosage sufficient to provide the desired result. For example, an effective amount may be an amount of the active agent present in the formulation that is sufficient such that, when applied to a skin site in accordance with the methods described herein, the subject's symptoms associated with Alzheimer's disease and/or dementia are treated.

In some embodiments, the transdermal active agent formulation may be applied to the skin site for an amount of time sufficient to deliver a target dose of the active agent to the subject over a period of time. For example, the target dose of the active agent may be 5 mg/day or greater, including 10 mg/day or greater, such as 15 mg/day or greater. In some cases, the transdermal active agent formulation may be applied to the skin site for an amount of time ranging from 1 day to 14 days, such as 3 days to 10 days, including 7 days to 10 days. In certain cases, the transdermal active agent formulation may be applied to the skin site for 7 days (i.e., one week).

After the transdermal active agent formulation has been applied to the skin site for the desired amount of time (i.e., an amount of time sufficient to deliver a target dose of the active agent to the subject over a period of time), the formulation may be removed from the skin site. A new transdermal formulation may be applied at the same or at a different skin site. The new transdermal formulation may be applied to a different skin site to reduce the possible occurrence of skin irritation and/or skin sensitization at the prior site of application.

In certain embodiments, the methods described herein may include a diagnostic step. Individuals may be diagnosed as being in need of the subject methods using any convenient protocol, and are generally known to be in need of the subject methods, e.g., they are suffering from a target disease condition or have been determined to be at risk for suffering from a target disease condition, prior to practicing the subject methods.

Diagnosis or assessment of Alzheimer's disease and dementia is well-established in the art. Assessment may be performed based on, but not limited to the following: patient history; collateral history from relatives; diagnostic tests, such as clinical observation of behavior; mental status testing of cognitive functions including but not limited to memory, language, perceptual skills, attention, constructive abilities, orientation, problem solving and functional abilities; physical examinations; neurological examinations; brain imaging, such as but not limited to computed tomography (CT), magnetic resonance imaging (MRI), positron emission tomography (PET), and single photon emission computed tomography (SPECT); and the like.

### Utility

The transdermal active agent systems find use in any application where a subject would benefit from being administered an anti-dementia active agent, such as but not limited to donepezil. In certain embodiments, the formulations are employed in the treatment of a condition. By treatment is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition.

In general, administration of donepezil according to the subject methods can be used to treat diseases or conditions including, but not limited to Alzheimer's disease, dementia, and the like. The transdermal active agent formulation may be used for administering donepezil to a subject. In these cases, the method includes applying a transdermal active agent formulation, as described herein, to a skin surface of a subject. The method further includes maintaining the active agent formulation on the skin of the subject for a period of time sufficient to deliver the active agent to the subject. Subjects may include humans or animals, such as but not limited to mice, rats, dogs, rabbits, and the like.

In certain embodiments, the transdermal active agent formulation is provided as an adhesive patch and is applied to the skin surface, whereby the active agent in the formulation can be administered by percutaneous permeation through the skin. When the transdermal active agent formulation is applied to a skin surface, the active agent permeates the skin in contact with the patch to reach the site of action through a systemic blood flow.

### Kits

Kits for use in practicing the methods described herein are also provided. In certain embodiments, the kits include components of the transdermal systems, e.g., a transdermal preparation, and first and second overlays. In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. In the subject kits, the one or more components are present in the same or different containers, as may be convenient or desirable.

The following examples are offered by way of illustration and not by way of limitation. Specifically, the following examples are of specific embodiments for carrying out the present invention. The examples are for illustrative purposes only, and are not intended to limit the scope of the present invention in any way.

### EXAMPLES

### I. Transdermal System

### A. Transdermal Preparation

A transdermal donepezil preparation having the format illustrated in FIG. 1 and further described in the Experimental Section of United States Published Patent Application 20070259028 (the disclosure of which is herein incorporated by reference) was prepared. The backing layer was a multi-laminate construct with polyethylene terephthalate (i.e., polyester) and pigmented polyethylene. The release liner was a polyester film with a silicone coating on both sides.

### B. First Overlay

The first overlay was a closed cell polyolefin foam overlay comprising a pressure sensitive acrylate adhesive.

### C. Second Overlay

The second overlay structure was a single-coated polyurethane medical tape on a white carrier.

### II. Application Protocol

In using the above transdermal system, the release liner is removed from the preparation and the adhesive layer is contacted with the skin site of interest. The first overlay is then applied over the patch, followed by the application of a second overlay structure configured to cover both the first overlay and the preparation.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A transdermal anti-dementia active agent system, said system comprising:
(a) a transdermal preparation comprising:
a backing;
an active agent reservoir layer comprising an anti-dementia active agent; and
an adhesive layer;
(b) a first overlay configured to at least partially cover the transdermal preparation when topically applied; and
(c) a second overlay configured to at least partially cover the first overlay when topically applied.

2. The system according to claim 1, wherein the first overlay is configured to entirely cover the transdermal preparation when topically applied.

3. The system according said claim 2, wherein the second overlay is configured to entirely cover the first overlay when topically applied.

4. The system according to claim 3, wherein the anti-dementia active agent is donepezil.

5. The system according to claim 4, wherein donepezil is present as a salt.

6. The system according to claim 5, wherein the donepezil salt is present in an amount ranging from 0.5% to 50% (w/w).

7. The system according to claim 1, wherein the active agent reservoir layer comprises:
an aminated polymer;
a polyhydric alcohol;
a carboxylic acid ester; and
an acrylic polymer.

8. The system according to claim 7, wherein the aminated polymer is a copolymer comprising a dialkylaminoalkyl(meth)acrylate and a monomer unit selected from an alkyl(meth)acrylate, a hydroxyalkyl(meth)acrylate, and a combination thereof.

9. The system according to claim 8, wherein the aminated polymer is a methyl(meth)acrylate-butyl (meth)acrylate-dimethylaminoethyl(meth)acrylate copolymer.

10. The system according to claim 7, wherein the carboxylic acid ester is selected from an ester of a polyvalent carboxylic acid and a monohydroxy alcohol, an ester of a fatty acid and a polyhydric alcohol, and a combination thereof.

11. The system according to claim 10, wherein the ester of a polyvalent carboxylic acid and a monohydroxy alcohol is an alkyl citrate ester and/or an alkyl sebacate ester.

12. The system according to claim 10, wherein said ester of a fatty acid and a polyhydric alcohol is at least the one selected from the group consisting of a sorbitan fatty acid ester, a propylene glycol fatty acid ester and a glycerin fatty acid ester.

13. The system according to claim 7, wherein the polyhydric alcohol is a sugar alcohol and/or a glycol.

14. The system according to claim 13, wherein the polyhydric alcohol is at least the one selected from the group consisting of glycerin, propylene glycol, dipropylene glycol, butylene glycol and polyethylene glycol.

15. The system according to claim 1, wherein the transdermal preparation comprises a rate-controlling membrane positioned between the active agent reservoir layer and the adhesive layer.

16. The system according to claim 1, wherein the adhesive layer comprises an acrylic polymer.

17. The system according to claim 16, wherein the adhesive layer comprises a carboxylic acid ester.

18. The system according to claim 17, wherein the carboxylic acid ester of the adhesive layer is selected from an ester of a polyvalent carboxylic acid and a monohydroxy alcohol, an ester of a fatty acid and a polyhydric alcohol, and a combination thereof.

19-20. (canceled)

21. The system according to claim 1, further comprising a release liner associated with the adhesive layer.

22-41. (canceled)

42. A structure comprising:
(a) a transdermal preparation comprising:
a backing;
an active agent reservoir layer comprising an anti-dementia active agent; and
an adhesive layer;
(b) a first overlay at least partially covering the transdermal preparation; and
(c) a second overlay at least partially covering the first overlay.
